(19) United States
(12) Patent Application Publication
(10) Pub. No.: US 2023/0160389 A1
(43) Pub. Date: May 25, 2023

(54) SUBMERSIBLE PUMP DETECTION SYSTEM

(71) Applicant: INDUSTRIAL FLOW SOLUTIONS OPERATING, LLC, New Haven, CT (US)

(72) Inventor: Mark Grzeika, Thomaston, CT (US)

(73) Assignee: INDUSTRIAL FLOW SOLUTIONS OPERATING, LLC, New Haven, CT (US)

(21) Appl. No.: 17/531,764

(22) Filed: Nov. 21, 2021

(51) Int. Cl. F04D 15/02; G01N 33/18; G01N 33/28; G01F 23/292; G01N 21/94

(52) U.S. Cl. CPC F04D 15/0218; G01N 33/1833; G01N 33/28; G01F 23/2928; G01N 21/94; G01N 2021/945

(57) ABSTRACT

The invention relates to an apparatus, system, and method for detecting a layer of contaminants, such as hydrocarbons, on the surface of accumulated water within a confined area that is periodically evacuated by a pump. The detection of contaminant layers may be accomplished through the use of an optical detection system comprising a light source, a plate having conductive, capacitive, positional and/or reflective properties, and a capacitive, pressure, optical, or ultrasonic sensor capable of distinguishing between oil and water, with the plate positioned at a distance equal to the desired minimum detectable hydrocarbon layer thickness. Dissipation of turbulence and agitation of the accumulated water may be achieved with a stilling tube. Additional sensors may detect high, intermediate, and low levels, as well as trigger optical sensor measurement. An integrated controller may determine the state of media within the confined area.

### TECHNICAL FIELD

The present disclosure relates generally to the control of pumps or pumping installations specially adapted for raising fluids at a depth and, in particular, to indicating or measuring liquid levels and indicating, measuring, or otherwise distinguishing between various fluid properties within a confined area that is periodically evacuated by a pump.

### BACKGROUND

Submersible pumps and pumping installations serve a variety of common applications. For example, water collection systems often require submersible pumps to avoid overflow and/or to move the water to a larger holding facility or processing station such as, for example, a water treatment filtration plant. Similarly, in low-lying confined areas having important technological infrastructure such as, for example, substations having electrical transformers, submersible pumps are used protect against environmental and/or other infrastructure damage resulting from unintended flooding of the confined area. In another example, commercial and residential building codes typically require construction of a sump and associated pumping systems for evacuating flood water from the site.

In these applications and others, fluids discharged by submersible pumps may have contaminants in the fluid such as, for example, rain water migrating from the source through the site to the location of the pump. Environmental contaminants including hydrocarbons, oils, and other hazardous substances are picked up by the rain water and disposed in the moat or other enclosure housing the pump associated installations. Similarly, rainwater runoff in urban areas is subject to contamination from hydrocarbons resulting from oily pavement, rain-washed refuse, or oily spills. The inadvertent mixture of water with oil are common contaminants associated with these applications and subsequent, unabated discharge represents a problem because of the potential harm caused to the environment and these installations may be strictly controlled by governmental regulations.

Several control system designs are known that ensure the submersible pump only pumps water out of the confined area, while leaving the oil within the confined area, so that it can be separately discharged, collected, or otherwise removed safely. These systems operate based on the physical properties of water and oil; namely, that water and oil form an immiscible mixture (i.e., an emulsion that tends to separate into distinct layers), where oil tends naturally to rise to the mixture's surface and form a separate layer from the water. As the inlet of a submersible pump is typically positioned toward the bottom of the pump system, the control system will work to maintain the oil layer above the pump's inlet. In this way, the submersible pump system removes the lower water layer as it accumulates, while ensuring that the water level remains high enough to keep the oil layer away from the pump's inlet and within the confined area.

To achieve the desired effect using this arrangement, known control systems employ sensors to detect the presence of water or oil at discrete locations within the confined area. Known solutions may use a mechanical flow switch (e.g., a float) combined with a conductive sensor and/or an optical sensor to operate a submersible pump. For optical sensors, the system may control based on distinguishable reflective properties of the respective media. In terms of conductivity sensors, the system may control based on distinguishable conductivities of the respective media.

However, these systems have proven to be problematic as a level-sensing device in terms of accuracy and/or precision, primarily because external factors promote turbulence (or agitation) of the media as it is deposited within the confined area surrounding the submersible pump. Turbulence may occur due to operation of the pump and/or environmental conditions including wind or rain. Turbulence may also occur based on the geometry of the confined area—as submersible pumps are typically offset below the surrounding area at a distance. This geometry (i.e., by the very nature of the system's geometric configuration), causes the liquid media to fall into the confined area from a sufficient height to induce mixing and turbulent conditions. The diameter or cross-sectional area of the confined area also impacts the degree to which the media may settle. Known systems may further be limited by the amount of contaminant that the system can detect; when turbulence exists, lesser amounts of contaminant may remain undetected, rendering only larger amounts detectable. This means that water levels corresponding to sensor locations where control values must be measured are inherently subject to undesirable conditions as it relates to precise sensor registration and overall system control. Improper accuracy and/or precision of the level-sensing in operations has problems in leaving hydrocarbons undetected and discharged to the environment or otherwise out of the system.

Furthermore, each type of sensor may present certain challenges. For one, mechanical floats/switches are relatively imprecise, as they operate based on a tilting motion, and therefore may trigger the associated electrical relay over a relatively wide water level range. The presence of turbulent water may further affect the precision of a float switch or other level measuring device. For another, conductivity sensors are negatively affected by turbulent conditions, as turbulence promotes mixing, which results in lack of oil detection and/or false readings. Similarly, optical sensors require relatively calm conditions to produce accurate and precise readings. This is because these sensors control, and are calibrated, based on the aforementioned distinguishable physical characteristics of the respective media.

For at least these reasons, a need exists to solve problems for submersible pump applications using known control systems that are subject to inaccurate readings, false alarms, or leaving hydrocarbons undetected and discharged to the environment.

Consequently, a long-felt need exists for submersible pump applications to provide a reliable, precise, and accurate contaminant detection control method of component of a submersible pump system for detecting hydrocarbons and not discharging contaminants outside the system or into the environment. Another long-felt need exists for a control device, system and method that is capable of operating under external conditions that cause turbulence or agitation of the media to be pumped out of the confined area surrounding the pump. Furthermore, a need exists for a modular, replaceable, and easily serviceably control device, system and method for detecting hydrocarbons and not discharging contaminants outside the system or into the environment.

### SUMMARY

The present disclosure provides an apparatus, system, and method for reliably, precisely, and accurately detecting a layer of a contaminant, like hydrocarbons, present with water in a confined area that is periodically evacuated by a pump capable of operating under external conditions that cause turbulence or agitation. The apparatus, system, and method for may be configured for the detection of other contaminants, besides hydrocarbons, that are of interest.

In one aspect of the present disclosure, one or more sensors may be located within a stilling pipe, capable of reducing turbulence or agitation to permit desired readings.

In another aspect of the present disclosure, the stilling tube may be electrically coupled to one or more sensors, the stilling tube being configured to complete a circuit within which different media may be sensed and/or distinguished, based on conductivity or other physical properties of the media.

In another aspect of the present disclosure, a sensing plate is optically coupled to an optical sensor, configured to detect a layer of water from a layer of hydrocarbons present on the surface of water and/or each of water or oil from air, for example, water and hydrocarbons present on the surface of the water from air.

In another aspect of the present disclosure, the apparatus, system, and method is capable of reliably and detecting the presence of a hydrocarbon layer with improved accuracy by measuring about 1/16 of an inch or 1.5875 millimeter, or by measuring from about 1/16 of an inch or 1.5875 millimeter to about 1/32 of an inch or 0.79375 millimeter, and/or by measuring amounts about 1/16 of an inch or 1.5875 millimeter and lower amounts under ideal conditions in a stilling tube, moat, and/or well.

In another aspect of the present disclosure, a sensing plate is electrically coupled to one or more sensors, the sensing plate being configured to complete a circuit within which different media may be sensed and/or distinguished, based on conductivity of the media.

In another aspect of the present disclosure, a control device, system and method using a programmable logic controller (PLC) electrically coupled to one or more sensors, a stilling tube, and/or a sensing plate, the PLC configured to provide reliable, precise, and accurate detection of a layer of contaminants, such as hydrocarbons.

In another aspect of the present disclosure, the controller circuit is configured to identify a change in a process variable as the input to a proportional-integral-derivative (PID) controller.

In another aspect of the present disclosure, the apparatus, system, and method includes a variable frequency drive (VFD) electrically coupled to a submersible pump to control the pump over an operable range, thereby reducing the energy required to operate the system.

In another aspect of the present disclosure, upon detecting a layer of contaminants, the apparatus, system, and method uses a controlled activation of one or more alarms to indicate the presence of the contaminants, pump failure, system component failure, or exceedingly high media level(s). Another aspect is to use a solid-state contactor for the controlled activation of alarms for the indicators monitored by the apparatus, system, and method

In another aspect of the present disclosure, the apparatus, system, and method provides remote monitoring capability, using cellular cloud-based monitoring, local network monitoring, and/or dry contact monitoring.

In yet another aspect of the present disclosure, upon detecting a layer of contaminants, the apparatus, system, and method controls a submersible pump so that the layer of contaminants remains above the pump inlet, while simultaneously allowing water to be evacuated from the confined area.

### DESCRIPTION OF DRAWINGS

Non-limiting and non-exhaustive embodiments of the present disclosure are described with reference to the following drawings. In the drawings, like reference numerals refer to like parts throughout the various figures unless otherwise specified.

For a better understanding of the present disclosure, reference will be made to the following Detailed Description, which is to be read in association with the accompanying drawings, which are incorporated in and constitute a part of this specification, show certain aspects of the subject matter disclosed herein and, together with the description, help explain some of the principles associated with the disclosed implementations, wherein:

FIG. 1 is a side view of a submersible pump detection apparatus, system, and method, in accordance with an embodiment of the present disclosure;

FIG. 2 is an enlarged view of the portion of FIG. 1, in accordance with an embodiment of the present disclosure;

FIG. 3A is a schematic side view of an optical sensor assembly illustrating an air condition, in accordance with an embodiment of the present disclosure;

FIG. 3B is a schematic side view of an optical sensor assembly illustrating a hydrocarbon condition, in accordance with an embodiment of the present disclosure;

FIG. 3C is a schematic side view of an optical sensor assembly illustrating a water condition, in accordance with an embodiment of the present disclosure;

FIG. 4 is a schematic diagram of signal processing circuitry for the submersible pump sensor architecture of FIG. 1; and

FIG. 5 is a side view of a submersible pump detection apparatus, system, and method, in accordance with an alternative embodiment of the present disclosure.

### DETAILED DESCRIPTION

Non-limiting embodiments of the invention will be described below with reference to the accompanying drawings, wherein like reference numerals represent like elements throughout. While the invention has been described in detail with respect to the preferred embodiments thereof, it will be appreciated that upon reading and understanding of the foregoing, certain variations to the preferred embodiments will become apparent, which variations are nonetheless within the spirit and scope of the invention.

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “some embodiments”, “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawings featured in the figures are provided for the purposes of illustrating some embodiments of the present disclosure, and are not to be considered as a limitation thereto. The term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein, the term “accuracy” refers to how closely individual measurements agree with the correct, or true, value.

As used herein, the term “confined area” refers to any defined area that may contain fluid media and which may be evacuated by an active process such as pumping, including but not limited to sumps, moats, transformer vaults, elevator shafts, and other equivalent structures.

As used herein, the term “precision” refers to a measure of how closely individual measurements agree with one another.

As used herein, the term “reliability” refers to a level of confidence in detecting contaminants based upon the acceptable calibration of the apparatus and/or system, and based upon the repeatability of the measurements obtained.

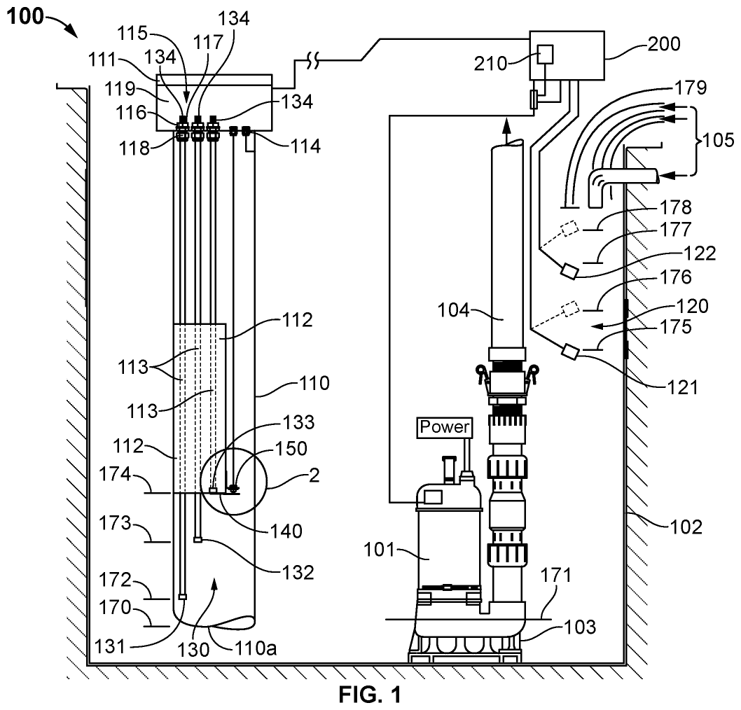

The submersible pump detection apparatus, system and method 100 of the present disclosure can be incorporated into a wide range of pumping installations and applications, can use other components and arrangements to achieve similar objectives, and is described in the context of a detection submersible pump detection system used in contaminant application as an exemplary embodiment as shown in FIGS. 1-5. The submersible pump detection apparatus, system and method 100 may use suitable sensors from the group of: capacitive sensor, pressure sensor, optical sensor, and ultrasonic sensor technology. For example, Capacitive level sensors operate on the property that the liquid is known as the dielectric using the key property of dielectric materials and the dielectric constant or relative permittivity with air having a dielectric constant of 1 (Air (at STP, for 0.9 MHz) is 1.00058986±0.00000050), e.g. diesel or transformer oils have a dielectric constant range of 2.1-2.5 and water (ranging from 80.1 at 20° C. to 10 at 360° C. This property is the amount of charge the liquid can absorb. Another example is that ultrasonic sensors transmit and receive high-frequency soundwaves to detect the presence of objects and can measure the level of liquids, solids, and/or powders using piezoelectric components configured to vibrate to produce the soundwaves, which are received by a transducer, so as to calculate contaminant levels or distance by measuring the time it takes for the signal to return to the sensor.

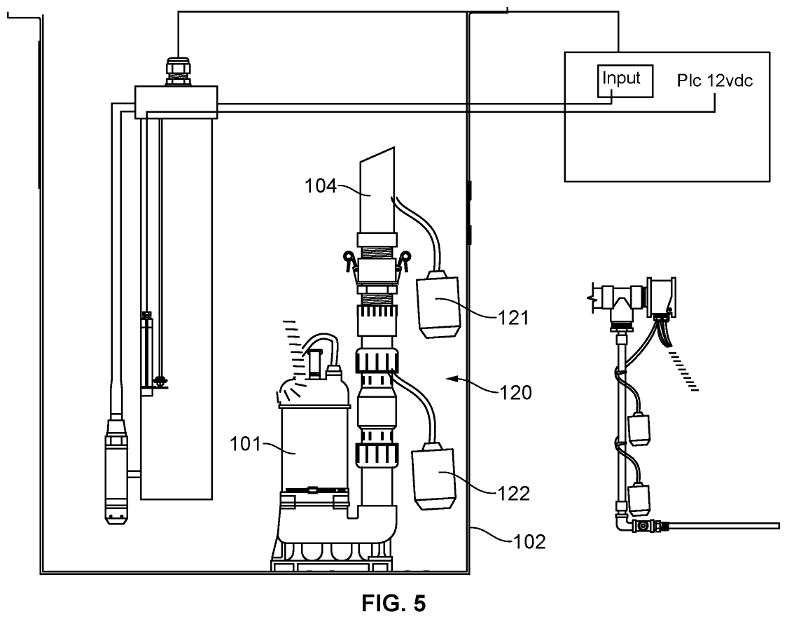

As shown in FIG. 5, the control module 200 may use an input signal from a pressure transducer to accurately monitor the level such as, for example, 4 to 20 milliamp pressure transducer. When a thin layer of hydrocarbons passes through the detection area, a PLC 210 will send a signal to a third sensor 133 VFD may be disposed at a third probe level 173 to maintain a level that would be considered safe so hydrocarbons cannot enter the suction area of the pump this is accomplished through a PID function on the drive itself.

FIG. 1 is a side view schematic of an exemplary preferred embodiment of a submersible pump detection system 100 having particular utility in a confined area and in conjunction with a pump capable of evacuating liquid media from the confined area using various system components such as piping, according to the present invention. Pumps typically employed for this purpose include, but are not limited to, submersible pumps and suction pumps. Furthermore, the submersible pump detection system 100 may be configured to control a single pump, or multiple pumps that serve the purpose of providing pump diversity, redundancy, or other similar purposes. For ease of illustration, FIG. 1, and subsequent depictions, show a single pump, although the submersible pump detection system 100 may control more than one pump.

Submersible Pump Detection System

As is illustrated in FIG. 1-4, a submersible pump detection apparatus, system, and method is generally shown as element 100. The pump detection system 100 is configured to be installed with a pump 101 and operated within a confined area 102 purposed to evacuate or otherwise pump accumulated water through pump inlet 103, located proximate a pump inlet level 171, through to discharge tube 104, while detecting the presence of a contaminant, like hydrocarbons, that may intermix with the accumulated water. As demonstrated here, the accumulated water may enter the confined area 102 through a confined area supply 105, which may be a pipe, grate, or any other mechanism that conveys accumulated fluids, liquids, and/or water from a source to the confined area 102. Advantageously, the pump detection system 100 detects or senses the presence of contaminants regardless of how turbulent or agitated the accumulated water may be within the confined area 102.

In one aspect of the present disclosure, pump detection system 100 may include a stilling tube 110, which may be configured to calm or otherwise dissipate turbulent conditions of the accumulated water within the confines of the stilling tube 110. To achieve this affect, a stilling tube inlet 110a may be located adjacent the lower portion of the confined area 102, as shown in FIG. 1, which is depicted as stilling tube inlet level 170. Level 170 is positioned, vertically with respect to the ground, and laterally with respect to the walls of confined area 102, so that it may accept accumulated water within the interior, but provide for turbulence-dissipating effects as quickly and effectively as possible. Depending on the application, stilling tube inlet level 170 may be positioned below, at, or above, the pump inlet level 171. Similarly, the geometry of stilling tube 110 may take any form to achieve the desired effect; for example, the cross-section of stilling tube 110 may be circular, square, rectangular, or elliptical. The cross-sectional area of stilling tube 110 may remain constant along its length, or may be tapered. Also, the cross-sectional area of stilling tube 110 may transform shape along its length such that one end takes the form of a first shape, such as a square, and the other end takes the form of a second shape, such as a circle. Additionally, stilling tube 110 may be formed of any thickness, uniform or tapered; in a preferred embodiment, the stilling tube 110 is uniformly ¼ inch thick.

Stilling tube 110 may further be configured with a purge opening located at an upper end thereof, to negate pressure effects of air contained within the interior of the stilling pipe 110. The purge opening would therefore allow air to enter and exit the stilling pipe as the accumulated water level changes, so that a pressure differential does not occur across the stilling pipe boundary, allowing the internal level and external level of the accumulated water to remain the same. The purge opening may alternatively be formed by the cumulative effect of openings provided by the system components disposed atop the stilling pipe 110. Additional openings disposed along portions of the stilling tube 110 may be included.

Furthermore, the stilling tube 110 may be configured to house one or more sensors 130, and/or one or more optical sensors 150 therein. As such, the cross-sectional area, as detailed above, may be further sized in any manner suited to achieve the dissipation of accumulated water, the housing of said one or more sensors 130, the housing of said one or more optical sensors 150, and the space required for the pump detection system 100 to operate reliably, accurately, and precisely. As further detailed below, the one or more sensors 130 and/or the one or more optical sensors 150 may be configured to reliably, accurately, and precisely detect the presence of contaminants, like hydrocarbons, present within the confined area 102. However, an objective of the stilling tube 110 is to promote an immiscible mixture to form (i.e., an emulsion of, for example a hydrocarbon layer on top of a water layer). This is largely achieved by separating the general accumulated water within the confined area from the localized accumulated water adjacent the sensors 130, 150, so that the localized water may settle, turbulent effects may substantially dissipate such as, for example, settling the mixture from foam to a non-emulsion mixture, and sensors 130, 150 may accurately and precisely register a reading of the conditions to determine whether contaminants are present

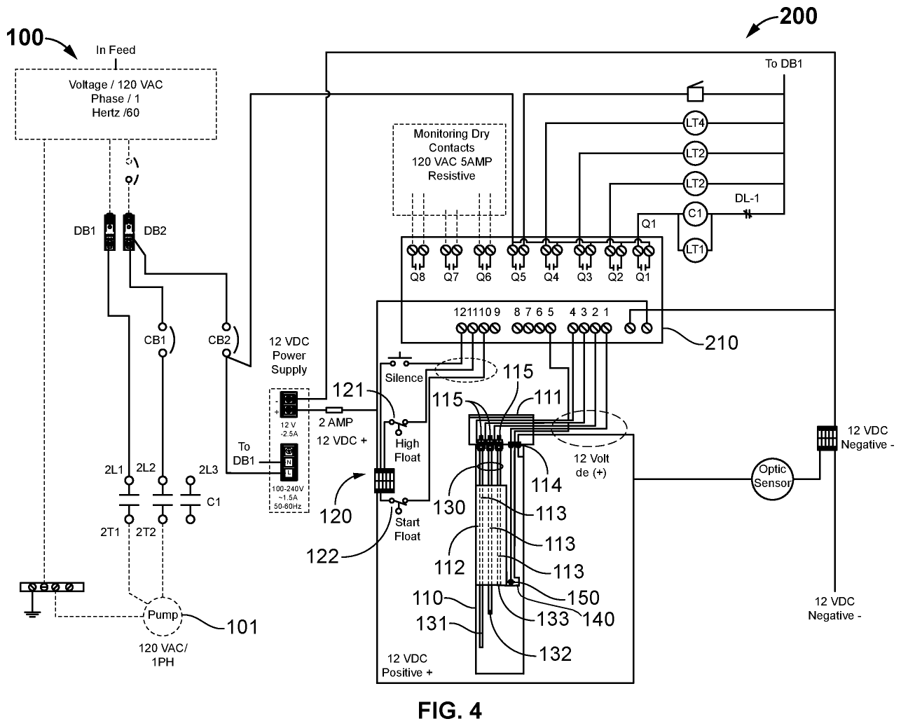

Stilling tube 110 may be further configured to electrically couple to a control module device 200. Stilling tube 110, therefore, may further facilitate registration and readings taken by one or more sensors 130. For example, in a preferred embodiment, stilling tube 110 is biased at +12V DC relative to the one or more sensors 130 via an isolated contact 114 connected to the stilling tube 110, as shown in FIGS. 1 and 4. Stilling tube 110 therefore may be made of a conductive material, including but not limited to, stainless steel, aluminum, zinc, and brass. Furthermore, the stilling tube 110 may comprise more than one material, so that only a portion of the stilling tube 110 is used for registering a physical property/value.

In another aspect of the present disclosure, pump detection system 100 may include a sensing plate 140. Sensing plate 140 may be configured to provide a surface upon which accumulated water may collect for the purposes of registering a reading from the one or more optical sensors 150. In accordance with this objective as shown in FIGS. 1-5, sensing plate 140 comprises a substantially flat upper surface disposed at an upper surface level 174′. According to one principle of operation wherein the sensing plate 140 is considered in isolation (i.e., irrespective of other pump detection system 100 components), the sensing plate 140 becomes saturated with the accumulated water level as it fills the confined area 102, where the accumulated water level rises above the upper surface level 174′. At a later point in time, the accumulated water level decreases, and falls below the upper surface level 174′. The sensing plate 140, due to the natural occurrence of surface tension of the deposited media (among other forces acting thereon), leaves a layer on the flat upper surface of sensing plate 140. With proper utilization of one or more optical sensors 150, as further detailed below, the deposited media may be analyzed according to one or more of its physical properties to render a decision as to whether contaminants are present in the accumulated water. Therefore, sensing plate 140 may take the form of any shape appropriate to achieve this desired objective, such as various cross-sectional areas, general geometric shapes (square, circular, and the like). This disclosure further contemplates any other shape of the sensing plate 140; for example, the upper surface may be convex or concave, or may have abrasions to promote the adhesion of media thereon. Additionally, sensing plate 140 may be oriented substantially flat with respect to the horizontal, or may be oriented at an angle relative to the horizontal; the optical sensor 150 and associated components may be similarly positioned such that an accurate and precise reading may be taken of the physical property under consideration.

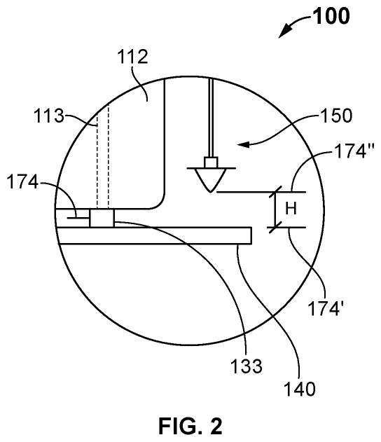

Sensing plate 140 may further be configured to electrically couple to a sensor 130, such as a third sensor 133 shown in FIGS. 1 and 2. In accordance with this objective, sensing plate 140 comprises a material having a sufficient conductivity for the sensor to register a reading through sensing plate 140, including but not limited, to stainless steel, aluminum, zinc, and brass. Furthermore, the sensing plate 140 may comprise more than one material, so that only a portion of the sensing plate 140 is used for registering a physical property/value. Alternatively, the sensing plate 140 may include geometric features that increase the surface area used to register a reading by the third sensor 133.

In another aspect of the present disclosure, pump detection system 100 may include a control module device 200, as shown in FIGS. 1, 4, and 5. As will be further detailed below, control module device 200 may be electrically coupled to one or more system components to achieve one or more of the operations contemplated in the present disclosure, including but not limited to: evacuating accumulated water; detecting contaminants, such as hydrocarbons; retaining contaminants by elevating and maintaining the accumulated water level above the pump inlet level 171, while simultaneously evacuating additionally accumulated water via discharge tube 104; turning on, off, or otherwise modulating the speed of the pump 101; enacting audible/visual alerts; and/or communicating with wireless network and remote control systems.

In the embodiment shown in FIG. 4, the control module device 200 comprises a 12V DC PLC 210 with sensing inputs and actuating outputs. The inputs may include the start float 121, high-level float 122, first, second, and third sensors 131, 132, 133, respectively, and one or more optical sensors 150. For a single float 120 embodiment, the float functions as a start float 121. In an optional float 120 embodiment, high level float 122 may be added for sensing an imminent overflow condition. The outputs include pump on/off relay, one or more audible alerts, and/or one or more visual indicators, for notifying the presence of hydrocarbons, a high-water level, and/or that the pump 101 is running as expected. As illustrated in FIG. 1, control module device 200 may be electrically coupled to the stilling tube 110, one or more sensors 130, one or more optical sensors 150, pump 101, and mechanical floats 120 (including start float 121 and high-level float 122). In operation, the start float 121 sensing an imminent overflow condition is configured for controlling the starting of the process to discharge water from the moat under control of the pump detection system 100, and the high level float 122 can be used for other control functions or as a confirmation signal for the starting the process the process to discharge water, if required.

Conductivity Sensor Assembly

As illustrated in FIGS. 1-5, stilling tube 110 may contain, within an interior space, one or more sensors 130. Sensors 130 may include first sensor 131, second sensor 132, and third sensor 133, disposed within stilling tube 110 according to the arrangement depicted in FIG. 1. As previously mentioned sensors 130 are disposed within stilling tube 110 in manner that allows for reliable, accurate, and precise registering of readings of one or more physical properties of media contained therein. Sensors 130 may be of any type capable of sensing a physical property of a medium. Sensors 130 may include, but are not limited to, capacitance-based sensors, conductivity probes, dielectric sensors, and/or combinations thereof. For example, said sensors 130 may be configured to detect the presence or absence of water and contaminants, like hydrocarbons (oil). First sensor 131 may be disposed at a first probe level 172, and may be configured and/or calibrated to sense a condition that prevents contaminants from entering pump inlet 103 by maintaining the accumulated water level above pump inlet level 171. Second sensor 132 may be disposed at a second probe level 173, and may be configured and/or calibrated to sense a condition that turns the pump 101 on, in the condition that a contaminant has previously been identified, so that, under normal operating conditions the contaminant level at its lowest level matches that of second probe level 173. Third sensor 133 may be disposed at a third probe level 173. Third sensor 133 may be configured to detect the presence of a hydrocarbon or other contaminant. As previously mentioned, third sensor 133 may be electrically coupled to sensing plate 140 to facilitate detection, among other purposes.

The one or more sensors 130 may further be coupled to, and insulated from, the stilling tube 110 wall by a PVC mounting block 112. The mounting block 112 is fitted with probe holes (rods) 113, which provide a press fit for the sensors 130. Electrically isolated feed-through assemblies 115 are disposed at the top of the stilling tube 110 for each of the sensors 130. Additional system components, as shown in FIG. 1, include: one or more insulated conductors 114, connected to said stilling tube 110; removeable access cover 111; nut 116; washer 117; grip connector 118; cable conductor 119; and threaded ends 134, to which sensors 130 may be affixed and ensure electrical coupling to the other parts of the pump detection system 100.

Under normal operating conditions, the pump will run when the start float 121 is activated and stop when the water level is pumped to the third sensor (stop probe) 131. Under adverse conditions, such as wind and rain, the surface of the water in the confined area 102 may be disturbed and deviate significantly from steady-state levels/conditions. Under such conditions, the accumulated water level sensing accuracy and precision would become compromised, but for the advantageous arrangement as substantially described and shown herein.

Optical Sensor Assembly

As shown in FIGS. 1-4, pump detection system 100 may comprise one or more optical sensors 150. Turning now to the enlarged view FIG. 2, taken from FIG. 1, certain particulars of an embodiment of pump detection system 100 may be seen. Optical sensor 150 may be disposed at an optical sensor level 174″, offset from sensing plate upper surface level 174′ of sensing plate 140, denoted as H. In a preferred embodiment, H is 1/16 of an inch; however, H may be any appropriate distance configured to detect media contained therein. Also shown in FIG. 2, third sensor 133 is shown at third probe level 174, wherein third sensor 133 is electrically coupled to sensing plate 140.

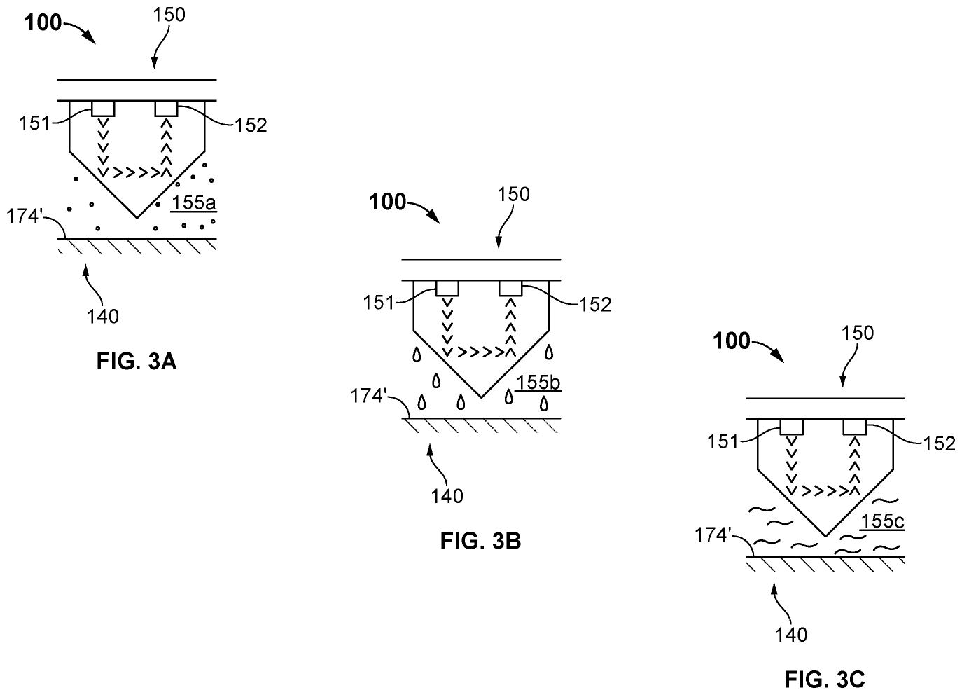

Turning now to FIGS. 3A-3C, the one or more optical sensors 150 are shown in various operating conditions indicative of the sensing condition contemplated within this disclosure as conditions typically observed by pump detection system 100. Common to FIGS. 3A-3C, one type of optical sensor 150 is shown having a transmitter 151 and a receiver 152, contained within a translucent housing. The translucent housing may take any shape configured to transmit, reflect, and receive an optical signal capable of calibrating based on unique properties of media, including but not limited to pyramidal and prismatic shape. Utilizing this configuration, along with the aforementioned conditions created by the sensing plate 140, the optical sensor 150 may be substantially saturated by the surrounding media, or medium, as depicted in FIGS. 3A-3C. In each of these illustrations, an optical signal is irradiated from the transmitter, a certain percentage is absorbed into the translucent housing, a certain percentage is transmitted through the surrounding medium, and a certain percentage of the optical signal will be reflected back to the receiver, which ultimately varies as a function of the surrounding medium's emissivity/absorbance.

As an air medium is nonconductive, and does not provide for dispersion of the light source, FIG. 3A shows a condition where air medium 155a is not detectable, and no signal and/or appropriate signaling is registered by control module device 200. Similarly, an oil medium is nonconductive, and does not provide for sufficient dispersion of the light source, FIG. 3B shows an alternative condition wherein a hydrocarbon medium 155b is not detectable and no signal and/or appropriate signaling is registered by control module device 200. Finally, as water is conductive, FIG. 3C shows yet another condition wherein a water medium 155c is detectable and appropriate signaling is registered by control module device 200. In this manner, optical sensor assembly 150 may detect, distinguish between, and provide appropriate signaling to said control module device 200 of the presence of a distinct medium. In this embodiment, optical sensor assembly 150 may do so for air, water and a contaminant, such as hydrocarbons.

Modes of Operation

In a first mode of operation, stilling tube 110, may be configured to maintain a level surface under all external conditions. In a second mode of operation, the stilling tube may be configured to sense the water level at three points: the stop level (first probe level 172), an intermediate stop level (second probe level 173), and an oil-sensing level (third probe level 174), which correspond to first, second, and third sensors 131, 132, and 133, respectively. In one embodiment, sensors 131, 132, 133 may detect a difference in conductivity between water (conductive) and hydrocarbons or air (insulative), thereby determining whether the surface of the water is above (high conductivity) or below (low conductivity) the sensor tip. The stop probe 131 may be set at a level (first probe level 172) that is proximate and above said pump inlet level 171, which ensures contaminants do not pass within pump inlet 103 of the pump 101. As previously mentioned, under normal operating conditions, the pump will run when the start float 121 is activated and stop when the water level is pumped to the third sensor (stop probe) 131.

A third mode of operation, stilling tube 110 and associated components of third sensor 133 and optical sensor 150 may be configured to detect hydrocarbon layers. This is achieved via the oil sensing level probe 140 and an optical sensor 150 which can detect the difference between water and hydrocarbons. A suitable optic sensor 150 is configured to sense water, oil, and/or anything that disperses the light source. If the optic sensor 150 does not distinguish between oil and water, the optical sensor 150 will generally activate through the introduction of media that cause dispersion of the optic sensor's light source. However, air and oil are nonconductive and cannot disperse the light source. If the light source is dispersed, PLC 150 input 2 is energized by z signal from the optical sensor 150 so as to turn on. If the light source 150 is not dispersed, oil is present, and a signal is not present at input 5, so as to turn off the PLC 150 due to loss of conductivity between the stilling tube 110 and the oil probe protruding plate 140. The system will then go into a hydrocarbon alarm.

The proper functioning of the optical sensor 150 requires that an optical signal pass through a homogeneous layer of liquid and be reflected into an optical detector. Thus, for a thin layer of hydrocarbons to be properly detected, it must fill the gap between the optical probe and the plate 140 that has a reflective surface and/or reflective properties. The ⅛″ thick plate 140 is factory set at a distance equal to the desired minimum detectable layer thickness, which may be 1/16″. In operation, as the pumping cycle occurs, the water level will drop below the oil-sensing-level probe 133, causing the input signal to change to a low state and triggering the optical sensor 150 to take a reading. This occurs quickly due to the high-speed programmable logic controller (PLC) 210 and its fast solid-state relay 160. If the gap between the sensor 150 and plate 140 is filled with hydrocarbons, the sensor will trigger the hydrocarbon alarm and pumping will be stopped immediately. If, however, water or air partially fills this gap, the hydrocarbon layer will not be accurately detected and the alarm indicating the presence of hydrocarbons will not be activated. This reinforces the need for a calm surface on the liquid to be sensed and the purpose of the stilling tube 110. Upon detecting a layer of hydrocarbons, the PLC will alter the pumping cycle to run between the start float 121 and the intermediate stop level probe 132, keeping the thin layer of oil at a safe distance from being discharged while still pumping water. The intermediate stop level probe 132 is factory set at a height that will facilitate keeping hydrocarbons battered by rain from entering through the water into the suction area of the pump.

Signal Processing Circuitry

FIG. 4 is a detailed schematic diagram of exemplary physical property sensor and actuation circuitry, in accordance with submersible pump detection system 100 that uses a physical property sensor in a manner that can detect hydrocarbons when the level is rising through the detection area or being pumped down through the detection area. Those skilled in the art will understand that in other embodiments, the signal processing can be partially or entirely contained and executed within the software of a microcontroller or other circuitry to achieve similar functionality as that identified herein; therefore, such alternatives do not depart from the scope of this disclosure. The physical property sensor can be selected from the group of electrical, electro-mechanical, optical sensors. For example, the optic sensor 150 can sense water and/or oil, and sense other substances that provides dispersion of the light source. The optic sensor 150 does not distinguish between oil and water it will activate with anything that can disperses the optic sensor's light source. Like oil, air is nonconductive, and cannot disperse the light source. If the light source is dispersed PLC input 2 turns on, oil present input 5 on the PLC goes off due to loss of conductivity between the stilling tube 110 and the oil probe protruding plate 140. The system will then go into a hydrocarbon alarm.

### Alternative Embodiments

In an alternative embodiment as illustrated in FIG. 5, stilling tube 110 may be fitted with a submersible continuous level transmitter in place of the two lower-level probes, first and second sensors 131, 132. A variable speed (VFD) pump and PID controller may be added to allow for a continuous liquid level maintenance mode wherein the water level is maintained within a small range.

In an alternative embodiment, communications hardware may be added to the system to effect internet connectivity for remote monitoring and/or control. The communications hardware may take the form of, for example, an ethernet adapter, a Wi-Fi adapter, or a cellular or other radio frequency transceiver.

In an alternative embodiment, the system may be fitted with solar power and battery backup.

The previous description of the disclosed embodiments is provided to enable any person skilled in the art to make or use the invention. Various modifications to these embodiments will be readily apparent to those skilled in the art, and the generic principles defined herein can be applied to other embodiments without departing from the spirit or scope of the invention. It is therefore desired that the present embodiments be considered in all respects as illustrative and not restrictive, reference being made to the appended claims as well as the foregoing descriptions to indicate the scope of the invention.

What is claimed is:

1. A submersible pump detection apparatus comprising:
a sensor in electric communication with a submersible pump, said sensor configured to detect a level of a fluid within a confined area; and
a stilling tube having a first opening disposed above a second opening, and a middle portion disposed therebetween, said sensor being disposed within said middle portion, wherein said stilling tube is configured to stabilize the fluid within an area proximate said sensor so that said sensor can register an accurate reading of a property of the fluid.

2. A submersible pump detection apparatus comprising:
a sensor in electric communication with a submersible pump, said sensor configured to detect a level of a fluid within a confined area; and
a sensing plate electrically and/or optically coupled to said sensor, said sensing plate including a surface having a longitudinal area capable of maintaining a fluid on said surface due to the surface tension of the fluid, such that said sensor may detect a property of the fluid.

3. A submersible pump detection apparatus comprising:
a sensor in electric communication with a submersible pump, said sensor configured to detect a level of a fluid within a confined area;
a sensing plate electrically and/or optically coupled to said sensor, said sensing plate including a surface having a longitudinal area capable of maintaining a fluid on said surface due to the surface tension of the fluid, such that said sensor may detect a property of the fluid; and
a stilling tube having a first opening disposed above a second opening, and a middle portion disposed therebetween, said sensor and said sensing plate being disposed within said middle portion, wherein said stilling tube is configured to stabilize the fluid within an area proximate said sensor so that said sensor can register an accurate reading of a property of the fluid.

4. A modular sensing unit comprising:
first, second, and third sensors, wherein each sensor is adapted to sense a property of one or more media at a distinct location, each sensor being disposed at a separate location to sense said one or more media and being at least partially embedded within an insulating block,
wherein said sensors further comprise a unitary device having plug-and-play connection, adapted to be installed and/or removed within a pump detection system.
